(19) United States
(12) Patent Application Publication
(10) Pub. No.: US 2019/0198174 A1
(43) Pub. Date: Jun. 27, 2019

(54) PATIENT ASSISTANT FOR CHRONIC DISEASES AND CO-MORBIDITIES

(71) Applicant: INTERNATIONAL BUSINESS MACHINES CORPORATION, Armonk, NY (US)

(72) Inventors: Soroush Abbaspour, Ossining, NY (US); Francisco P. Curbera, Hastings On Hudson, NY (US); Daniel M. Dias, Mohegan Lake, NY (US); Shahram Ebadollahi, White Plains, NY (US); Maria Eleftheriou, Mount Kisco, NY (US)

(21) Appl. No.: 15/853,297

(22) Filed: Dec. 22, 2017

(51) Int. Cl. G16H 50/30; G16H 10/60; G16H 30/20

(52) U.S. Cl. CPC G16H 50/30; G16H 20/10; G16H 30/20; G16H 10/60

(57) ABSTRACT

Patient assistant systems are provided. In various embodiments, health data of a user is read from one or more data source. A cohort of the user is determined based on a primary diagnosis of the user. The health data of the user includes the primary diagnosis. A co-morbidity of the primary diagnosis within the cohort is determined. One or more predictor of the co-morbidity within the cohort is determined. Assistance information is provided to the user based on the one or more predictor. The assistance information includes the predictor and one or more recommendation to mitigate the co-morbidity.

### BACKGROUND

Embodiments of the present disclosure relate to providing patient assistance information, and more specifically, to a patient assistant for chronic diseases and co-morbidities.

### BRIEF SUMMARY

According to embodiments of the present disclosure, methods of and computer program products for providing patient assistance information are provided. In various embodiments, health data of a user is read from one or more data source. A cohort of the user is determined based on a primary diagnosis of the user. The health data of the user includes the primary diagnosis. A co-morbidity of the primary diagnosis within the cohort is determined. One or more predictor of the co-morbidity within the cohort is determined. Assistance information is provided to the user based on the one or more predictor. The assistance information includes the predictor and one or more recommendation to mitigate the co-morbidity.

### BRIEF DESCRIPTION OF THE SEVERAL VIEWS OF THE DRAWINGS

FIG. 1 illustrates a system for providing patient assistance information according to embodiments of the present disclosure.

FIG. 2 illustrates a method for providing patient assistance information according to embodiments of the present disclosure.

FIG. 3 illustrates a method for providing patient assistance information according to embodiments of the present disclosure.

FIG. 4 depicts a computing node according to an embodiment of the present invention.

### DETAILED DESCRIPTION

The present disclosure provides for patient-focused analysis of a current condition and possible progression of co-morbidities. In various embodiments, guidance is provided to patients to help avoid progression of a chronic disease into additional co-morbidities and to assist in reversing existing chronic conditions.

For example, a patient diagnosed with type-2 diabetes has a higher probability of developing co-morbidities such as hypertension, heart failure, and chronic kidney disease. From longitudinal data of this and other patients, the probability of possible progressions of such co-morbidities can be estimated. Plans to mitigate such a progression can be defined and tracked on a patient mobile or IoT device.

This patient-focused approach provides customized guidance that is not possible in systems that focus only on population health. Similarly, the health context available through cohort analysis enables richer feedback to a user than merely tracking health parameters through a health or wellness application.

In various embodiments, a cohort is identified to which an individual patient belongs. The cohort may be based on similar conditions or history. Based on longitudinal data records over a period of time, the co-morbidities that this cohort of patients typically develop are analyzed. The predictors of these co-morbidities are determined, and potential actions to prevent such a progression are identified. Assistance is provided to the patient, for example in defining preventative actions, tracking performance of those actions, and in overall management of the patient's health and wellness.

In various embodiments, patient consent is obtained, for example through a digital consent form. Based on the consent from the patient, patient data is extracted from one or more EMR. Third party longitudinal data may also be extracted, where it exists, for example from IBM Explorys, Truven Health, or IBM Phytel. Based on longitudinal data from this cohort and spatio-temporal features (e.g., location, weather, pollution, etc.), the potential progression of co-morbidities is estimated, and predictions for this progression is determined. From knowledge data about the patient condition combined with the predicted progression, a digital patient assistant provides progression information to the patient, along with recommendations to mitigate the co-morbidities from developing. In various embodiments, specific items to monitor, such as exercise, diet, or environmental factors are provided to the patient and monitored by the system.

An electronic health record (EHR), or electronic medical record (EMR), may refer to the systematized collection of patient and population electronically-stored health and wellness information in a digital format. These records can be shared across different health care settings and may extend beyond the information available in a PACS. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

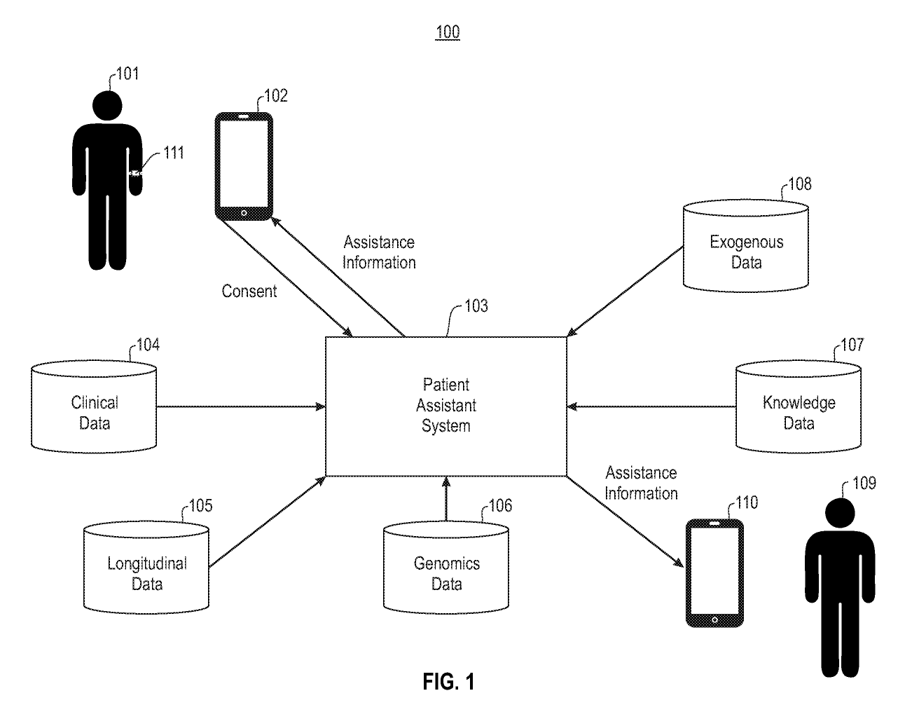

Referring now to FIG. 1, a system for chronic disease management is illustrated according to embodiments of the present disclosure. Patient 101 may use computing device 102 to provide consent for information disclosure to patient assistant system 103. Computing device 102 may be a mobile device or other computing node. Patient 101 may access system 103 via a web application, a mobile app, or other application method known in the art.

System 103 retrieves data from one or more data source relevant to patient 101. In various embodiments, the data sources may include clinical data 104, such as EMR, images, pharmacy data, or claim data. The data sources may include longitudinal data 105, for example from IBM Explorys, Truven Health, or IBM Phytel. Longitudinal data may include patient data from multiple EMR systems associated with multiple hospital or physician systems. The data sources may include genomics data 106, knowledge data 107, or exogenous data 108. Knowledge data 107 may include various sources of medical knowledge, official medical treatment guidelines, position papers, and the like, for a variety of medical maladies, such as chronic diseases extracted from patient care plan guidelines and knowledge sources. Exogenous data 108 may include environmental conditions, e.g., weather conditions, allergen level information, pollution levels, or other factors existing outside the patient's body that may affect a patient's chronic medical conditions.

As set forth below, assistance information for managing chronic diseases and co-morbidities may be generated based on the various patient data. Such assistance information may be provided to patient 101 via computing device 102. Likewise, assistance information may be provided to physician 109 via computing device 110.

In various embodiments, clinical data 104 or exogenous data 108 may be drawn from mobile device 102 or wearable device 111 of patient 101, or another computing device or sensor. For example, biometric data of patient 101 may be gathered, including, for example, heart rate, motion, blood sugar, or blood oxygen. Likewise, exogenous data may be gathered, including, for example, ambient temperature, humidity, barometric pressure, or other environmental data for patient 101.

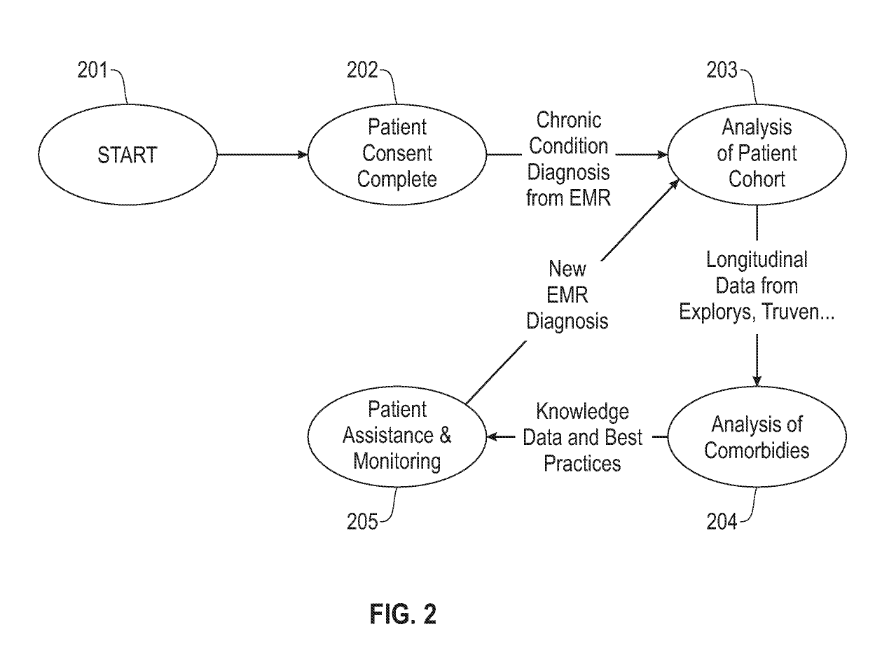

Referring now to FIG. 2, a method for providing patient assistance information for management of chronic conditions and co-morbidities is illustrated according to embodiments of the present disclosure. At 201, consent from the patient is obtained for collecting patient data from various data source. Data sources may include clinical data from EMRs, image data from PACS and Vendor Neutral Archives (VNAs), prescriptions and claims, exogenous data, or genomics data from sequencing or specific assays. In various embodiments, exogenous data may include, e.g., spatio-temporal information, such as weather, location, or pollution. In various embodiments, the permissions granted by patient 101 may relate to individual sources of data, or to individual types of data within a given data source. Permissions granted by patient 101 may also broadly pertain to all related data sources. In various embodiments, permissions may relate to individual monitoring devices, such as wearable activity, blood pressure, or glucose monitors.

Permissions granted by the patient may also include identification of authorized receivers of assistance data, and types of assistance data requested. For example, a patient may designated themselves and a caregiver as recipients of assistance data. It will be appreciated that consent may be gathered through a variety of methods, including distributed permission systems, and various access control methods known in the art.

Based on the patient consent, the patient assistant system accesses patient EMR data and retrieves a patient diagnosis and longitudinal information. The system then determines additional features of the patient diagnosis, including progression of condition, additional co-morbidities if any, and possibly related genomics information. Combining this with information from knowledge sources, parameters determining a cohort of patients are defined at 203.

Based on the specifications of the cohort containing the patient, de-identified data is extracted and a dataset is created for analysis of the patient's cohort. As noted above, such data may be retrieved from various sources of longitudinal data such as Explorys, Truven, or Phytel.

This cohort information may then be analyzed at various levels of detail. Typical characteristics of the cohort may be determined and provided to the patient. For example, cohort summary statistics may be derived such as for BMI, hypertension, diabetes, depression, chronic kidney disease (CKD), or other patient attributes or conditions. Such summary statistics may be presented to a patient to help them understand their condition. For example, data visualization such as histograms may be provided.

At 204, more detailed analysis of co-morbidities in the cohort are analyzed. In some embodiments, incidence of co-morbidities within a cohort group is determined, and may then be displayed to the user through a visualization such as a histogram. For example, the incidence of diabetes and depression for a cohort group may be displayed to a patient. In another example, the incidence of diabetes and chronic kidney disease (CKD) may be provided.

In some embodiments, further analysis is performed to determine predictors for poor control of the primary chronic condition of the patient. Similarly, predictors for progression to co-morbidities may be determined. For example, predictors for poor control of diabetes may be determined, such as lack of health insurance, using more than one oral hypoglycemic agent, obesity, or non-adherence to diabetic medications. In other examples, predictors for CKD, hypertension, depression, or other conditions are determined. These predictors may be presented to a patient, for example, through a chart indicating the relative correlation of each predictor to a given co-morbidity.

It will be appreciated that a variety of methods may be used according to the present disclosure to analyze predictors for progression to a co-morbidity based on longitudinal data.

In some embodiments, statistical analysis of correlation between occurrence of co-morbidities is performed, conditioned on the co-morbidity occurring after the primary diagnosis in the longitudinal data. Accordingly, longitudinal data is analyzed for subsequently occurring co-morbidities associated with primary diagnosis. The correlation of parameters and their predictive strength may be assessed using algorithms known in the art, including those provided in analysis packages such as IBM SPSS. For example, generalized linear mixed models (GLMM) or generalized linear models (GLM) may be applied. It will be appreciated that a variety of statistical models may be suitable.

Such analysis of may not establish causative factors, but predictive strength can be used to assess the importance of a parameter or combination of parameters within the patient cohort in controlling progression to co-morbidities. Those skilled in the art will readily appreciate that other techniques can be used in the analysis of predictors, such as machine learning algorithms, decision trees, regression, classification, support vector machines, k-nearest neighbors, neural networks, or other learning systems.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Following the analysis of co-morbidities 204, patient assistant systems according to the present disclosure provides patient assistance and monitoring 205. In this phase, results of the analysis are presented to the patient. In some embodiments, results are also provided to a physician or other care-giver. In some embodiments, results are provided to a patient via a mobile app, web application, or other computer output.

In some embodiments, analysis of patient data is continuous. For example, in some embodiments, ongoing biometric readings are collected. In some embodiments, patient-entered data such as meals consumed or activities performed are collected. In such embodiments, patient assistance information may be presented upon any significant change. A change in a parameter that is a factor in a co-morbidity, new EMR data, or new knowledge data may trigger a patient assistance message. For example, a new guideline being published (e.g., recent updates to guidelines on hypertension) may cause an update in patient assistance.

Based on the patient consent, the patient is presented with information on the patient's clinical, genomics, and exogenous data. It will be appreciated that the extent of the consent provided by the patient and the data actually collected will determine what additional information is provided to a user. Based on knowledge information, the patient is presented with information related to the patient's chronic condition, pharmaceutical side effects and other information related to the diagnosed condition and genetic information.

Knowledge data may be stored in a knowledgebase, and may comprise one or more explicit representation of knowledge. For example, in some embodiments, knowledge is embodied in a plurality of rules within a knowledgebase or rulebase. Knowledge data may include information on pharmaceutical interactions, pharmaceutical applicability to conditions, relationships among conditions, clinical guidelines regarding care and treatment, and various other domain specific information. It will be appreciated that expert knowledge can be incorporated into Bayesian networks, in addition to other approaches known in the art.

In various embodiments, a patient or practitioner is provided information related to data from a cohort of similar patients. This information may be de-identified, or presented only in aggregate. Cohort data may be obtained as outlined above. The patient may also be provided with information on how the patient's key health parameters related to this cohort. For example, a higher than average blood pressure within the patient's cohort may be flagged to the patient or to the care-giver. Finally, the patient is presented with recommendations (this could be done together with the care-giver) on health and wellness, nutrition and other actions based on the patient's condition relative to the cohort and predictors to prevent progression of/to co-morbidities.

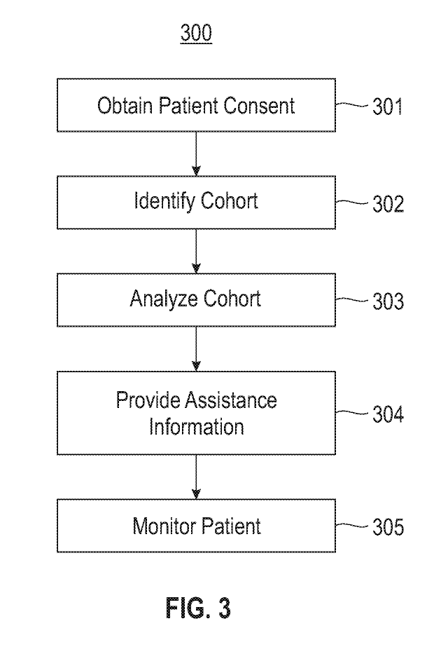

Referring now to FIG. 3, a method is illustrated for providing patient assistance for chronic diseases and co-morbidities according to embodiments of the present disclosure. At 301, consent is obtained from a user for gathering health data. In some embodiments, the user is a patient. In some embodiments, the health data includes health data of the patient. In some embodiments, health data includes clinical data, EMR data, genomic data, exogenous data, or knowledge data. In some embodiments, user consent is also obtained for presenting health data to the user or to others. In some embodiments, consent is obtained through a prompt within an desktop application, a mobile app, or a web application. Based on the consent, health data for the user is read from one or more data source.

At 302, a cohort is identified for the user. In some embodiments, the cohort is based on a primary diagnosis of the user. In some embodiments, the cohort is based on one or more heath attributes of the user. In some embodiments, the health attributes include age, body mass index, or genetic attributes. In some embodiments, the cohort is based on one or more external attributes of the user, such as location. In some embodiments, the cohort is identified within a dataset covering a plurality of patients. In some embodiments, the dataset is de-identified.

At 303, the cohort is analyzed to determine distributions of a plurality of health parameters. In some embodiments, the cohort is also analyzed to determine distributions of co-morbidities for the primary diagnosis. In some embodiments, one or more predictor for progression of the primary diagnosis to co-morbidities is determined. In some embodiments, the one or more predictor is determined by statistical analysis. In some embodiments, the one or more predictor is determined by application of a learning system. In some embodiments, the predictor comprises one of the plurality of health parameters. In some embodiments, the predictor comprises a time delay between the primary diagnosis and the co-morbidity within the cohort.

At 304, assistance information is present to the user. In some embodiments, the assistance information comprises summary data regarding health attributes of the cohort. In some embodiments, the assistance information comprises incidence of co-morbidities within the cohort. In some embodiments, the assistance information comprises predictors of progression of a co-morbidity. In some embodiments, the assistance information comprises a recommendation to mitigate one or more of the predictor of progression. For example, where a predictor is a health attribute, the recommendation may identify the health attribute. The recommendation may also identify one or more mitigation activities associated with the health attribute based on knowledge information. For example, where a high BMI is a predictor of progression of a co-morbidity, the recommendation may include exercise.

At 305, the patient is monitored with regard to one or more predictor of progression of a co-morbidity. In some embodiments, ongoing monitoring is provided via a mobile or wearable device. In some embodiments, a plurality of patient health parameters are monitored. In some embodiments, these monitored parameters are used to update the patient data. In some embodiments, patient consent is used to determine who has access to patient monitoring data.

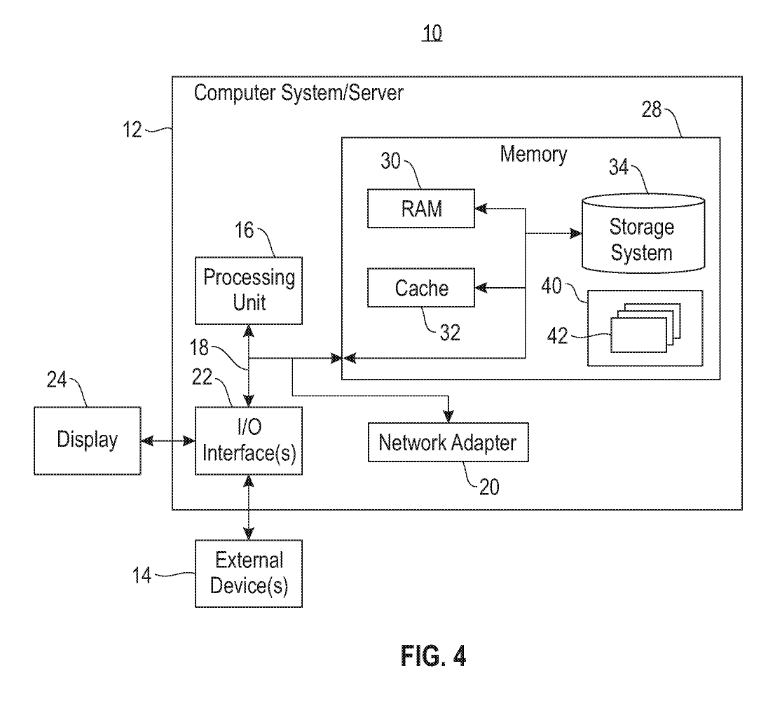

Referring now to FIG. 4, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
reading, by a processor, health data of a user from one or more data source;
determining, by the processor, a cohort of the user based on a primary diagnosis of the user, the health data of the user comprising the primary diagnosis;
determining, by the processor, a co-morbidity of the primary diagnosis within the cohort;
determining, by the processor, one or more predictor of the co-morbidity within the cohort;
providing assistance information to the user based on the one or more predictor, the assistance information comprising the predictor and one or more recommendation to mitigate the co-morbidity.

2. The method of claim 1, wherein the cohort is determined based on one or more heath attribute of the user.

3. The method of claim 1, wherein determining the co-morbidity comprises analyzing the cohort.

4. The method of claim 1, wherein determining the co-morbidity comprises applying knowledge data.

5. The method of claim 1, wherein determining the one or more predictor comprises applying a learning system.

6. The method of claim 1, wherein determining the one or more predictor comprises statistical analysis of the cohort.

7. The method of claim 1, further comprising:
obtaining consent from the user to gather and/or share health data of the user.

8. The method of claim 1, wherein the health data of the user comprise clinical data, EMR data, genomic data, exogenous data, or knowledge data.

9. The method of claim 1, further comprising:
monitoring health parameters of the user; and
providing updated assistance information upon change in the monitored health parameters of the user.

10. The method of claim 9, wherein the monitoring is performed by a wearable device of the user.

11. The method of claim 1, wherein the health data of the user comprise knowledge data, the method further comprising:
monitoring the knowledge data; and
providing updated assistance information upon change in the knowledge data.

12. A system comprising:
a computing node comprising a computer readable storage medium having program instructions embodied therewith, the program instructions executable by a processor of the computing node to cause the processor to perform a method comprising:
reading health data of a user from one or more data source;
determining a cohort of the user based on a primary diagnosis of the user, the health data of the user comprising the primary diagnosis;
determining a co-morbidity of the primary diagnosis within the cohort;
determining one or more predictor of the co-morbidity within the cohort;
providing assistance information to the user based on the one or more predictor, the assistance information comprising the predictor and one or more recommendation to mitigate the co-morbidity.

13. A computer program product for providing patient assistance information, the computer program product comprising a computer readable storage medium having program instructions embodied therewith, the program instructions executable by a processor to cause the processor to perform a method comprising:
reading health data of a user from one or more data source;
determining a cohort of the user based on a primary diagnosis of the user, the health data of the user comprising the primary diagnosis;
determining a co-morbidity of the primary diagnosis within the cohort;
determining one or more predictor of the co-morbidity within the cohort;
providing assistance information to the user based on the one or more predictor, the assistance information comprising the predictor and one or more recommendation to mitigate the co-morbidity.

14. The computer program product of claim 13, wherein the cohort is determined based on one or more heath attribute of the user.

15. The computer program product of claim 13, wherein determining the one or more predictor comprises applying a learning system.

16. The computer program product of claim 13, wherein determining the one or more predictor comprises statistical analysis of the cohort.

17. The computer program product of claim 13, the method further comprising:
obtaining consent from the user to gather health data of the user.

18. The computer program product of claim 13, wherein the health data of the user comprise clinical data, EMR data, genomic data, exogenous data, or knowledge data.

19. The computer program product of claim 13, the method further comprising:
monitoring health parameters of the user; and
providing updated assistance information upon change in the monitored health parameters of the user.

20. The computer program product of claim 13, wherein the health data of the user comprise knowledge data, the method further comprising:
monitoring the knowledge data; and
providing updated assistance information upon change in the knowledge data.
